(19) United States
(12) Patent Application Publication
(10) Pub. No.: US 2025/0073174 A1
(43) Pub. Date: Mar. 6, 2025

(54) BIODEGRADABLE ALGINATE FILMS

(71) Applicant: NUTRITION & BIOSCIENCES USA 1, LLC, Rochester, NY (US)

(72) Inventors: MATTHIAS KNARR, BOMLITZ (DE); OLIVER PETERMANN, BOMLITZ (DE)

(21) Appl. No.: 18/292,993

(22) PCT Filed: Jul. 28, 2022
(86) PCT No.: PCT/EP2022/071205

§ 371 Date: Jan. 29, 2024

(30) Foreign Application Priority Data
Jul. 29, 2021 (EP) 21188465.5

(51) Int. Cl. A61K 9/48

(52) U.S. Cl. CPC A61K 9/4816

(57) ABSTRACT

The present invention relates to water-insoluble micro-dispersions of calcium alginate, and in particular for water-insoluble micro-dispersions for the preparation of capsules and capsule films as enteric coatings for oral administration of medications, nutritional components and supplements.

### FIELD OF THE INVENTION

The present invention relates to water-insoluble micro-dispersions, and in particular to water-insoluble micro-dispersions for the preparation of capsules and capsule films as enteric coatings for oral administration of medications, nutritional components and supplements.

### BACKGROUND OF THE INVENTION

Biodegradable polymers are of high interest due to the upcoming ban of non-biodegradable plastics in the EU. Biodegradable polymers which are insoluble in water is of particular high interest. This because insolubility in water is required for several applications like delivery systems in agriculture or encapsulation in personal care applications.

Various esterified cellulose ethers are known as enteric polymers for pharmaceutical dosage forms, such as methylcellulose phthalate (MCP), hydroxypropyl methylcellulose phthalate (HPMCP), methylcellulose succinate (MCS), or hydroxypropyl methylcellulose acetate succinate (HPMCAS). The esterified cellulose ethers are well known for coating dosage forms, such as tablets, microparticulates or capsules. These enteric polymers protect the drug from inactivation or degradation in the acidic environment or prevent irritation of the stomach by the drug but are dissolved in the intestinal canals to release the drug contained therein.

These known enteric coatings or capsules are prepared from organic or aqueous compositions comprising esterified cellulose ethers. However, organic solvents are not desirable for pharmaceutical or nutritional uses due to high production costs and potentially remaining amounts of organic solvents in the esterified cellulose ethers. On the other hand, esterified cellulose ethers only have a limited solubility in water and there seem to be significant challenges in preparing dispersions with esterified cellulose ethers.

In view of the known disadvantages of organic and aqueous solutions of esterified cellulose ethers, much research effort has been spent on alternative and improved aqueous dispersions of enteric polymers.

Calcium alginate also referred to as calcium salts of alginic acid is a well-known biodegradable water insoluble biopolymer. For calcium alginate film formation sodium alginate solutions are being crosslinked with solutions containing calcium ions followed by drying and posttreatment with additional calcium ions containing solutions. This is a complex time-consuming multi-step process.

One example of the preparation of calcium alginate films by a prior art process is described in Braz. J. Chem. Eng. vol. 30 no. 4 São Paulo October/December 2013.

Accordingly, there is a need in the field for improved methods for film formation, and in particular for the preparation of capsules, such as for enteric use.

### BRIEF DESCRIPTION OF THE DRAWINGS

FIG. 1. Calcium alginate films from microdispersion with 100 wt-% glycerol relative to the amount of calcium alginate.

FIG. 2. Calcium alginate film (100% glycerol based on polymer) in water after 14 days at RT.

FIG. 3. Calcium alginate dry film separated from glass plate as prepared according to example 3.

FIG. 4. Calcium alginate dry 9.15% film separated from glass plate as prepared according to example 3.

### SUMMARY OF THE INVENTION

It is an object of embodiments of the invention to provide water-insoluble micro-dispersions suitable for the preparation of films, such as capsule films. Capsule films provided be the present invention may be used in the preparation of coatings of a dosage form suitable for enteric application, i.e. oral administrations wherein the drug, supplement or nutritional component is protected from inactivation or degradation in the acidic environment of the stomach or prevent irritation of the stomach by the drug, supplement or nutritional component but are dissolved in the intestinal canals to release the drug, supplement or nutritional component contained in e.g. an enteric capsule.

Accordingly, in a first aspect the present invention relates to a water-insoluble micro-dispersion comprising a) dispersed particles of calcium alginate in an amount of about 1% to about 20% by total weight of the micro-dispersion, and b) a plasticizer from at least about 30% to not more than about 200% by weight of the calcium alginate, and c) water in an amount of at least about 50% to not more than about 98% by total weight of the micro-dispersion.

In a second aspect the present invention relates to a method for the preparation of a water-insoluble micro-dispersion comprising the steps of combining dispersed particles of calcium alginate in an amount of about 1% to about 20% by total weight of the micro-dispersion with a plasticizer from at least about 30% to not more than about 200% by weight of the calcium alginate, and water in an amount of at least about 50% to not more than about 98% by total weight of the micro-dispersion.

In a third aspect the present invention relates to a process for coating a dosage form comprising the steps of a) preparing the water-insoluble micro-dispersion according to the present invention, b) optionally adding a further film forming aid or adjuvants to form an aqueous coating composition and c) coating a dosage form with the aqueous coating composition.

In a further aspect the present invention relates to the use of a water-insoluble micro-dispersion according to the present invention for encapsulation of a drug, a nutritional or food supplement, or a combination thereof. In some embodiments the use is for encapsulation of a drug, a nutritional or food supplement, or a combination thereof intended for enteral administration, such as enteric oral applications.

In a further aspect the present invention relates to a capsule film made from the water-insoluble micro-dispersion according to the present invention. In some specific embodiments the film has a thickness of from about 20 μm to about 200 μm, such as from about 40 μm to about 150 μm, such as from about 50 μm to about 120 μm. In some specific embodiments the film is at least 90% dissolved within 10 min to about 40 min, such as within about 20 min to about 30 min in a United States Pharmacopoeia (USP) phosphate buffer pH 6.8.

In a further aspect the present invention relates to a capsule comprising a capsule film according to the present invention, further comprising a drug or a nutritional or food supplement or a combination thereof.

In a further aspect the present invention relates to a process for producing capsule film comprising the steps of providing the water-insoluble micro-dispersion according to the present invention, pre-heating moulding pins to a temperature higher than the aqueous composition, dipping the pre-heated moulding pins into the water-insoluble micro-dispersion, forming a film on said moulding pins by withdrawing said pins from said water-insoluble micro-dispersion, and drying the film on the moulding pins.

### DETAILED DESCRIPTION OF THE INVENTION

The present inventors have found that a micro-dispersion of calcium alginate containing a plasticizer is capable of forming a firm and flexible calcium alginate film. The calcium alginate film is still insoluble in water. The new process should enable film coating and encapsulation applications by using a pre-produced ready to use micro-dispersion of calcium alginate.

It is very surprising that a cross-linked polymer like calcium alginate is capable of film forming by coalescence.

Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and G blocks. Gel formation at neutral pH requires a calcium source to provide calcium ion to interact with G-blocks. The greater the proportion of these G-blocks, the greater the gel strength.

“Alginate” is the term usually used for the salts of alginic acid, but it can also refer to all the derivatives of alginic acid and alginic acid itself; Alginate is present in the cell walls of brown algae (Phaeophyceae sp.) as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the alginate from the initial extraction solution is to add a calcium salt. This causes calcium alginate to form with a fibrous texture; it does not dissolve in water and can be separated from it. The separated calcium alginate is suspended in water and acid is added to convert it into alginic acid. This fibrous alginic acid is easily separated, placed in a planetary type mixer with alcohol, and calcium carbonate is gradually added to the paste until all the alginic acid is converted to calcium alginate. The paste of calcium alginate is sometimes extruded into pellets that are then dried and milled.

Alginates suitable for use in the practice of this invention will typically have a molecular weight such that they exhibit a viscosity in the range of 50-1,000 mPa·s. when measured at 1 wt % at 20° C. using Brookfield type RV (e.g. RVT, RVF, RVTDV) with Brookfield RV using the appropriate spindle for the viscosity range in question. The appropriate spindle for the viscosity determination can be readily determined by one of ordinary skill in the art, based on the equipment model and the viscosity range. In some embodiment, such alginates will exhibit a viscosity of between 30 and 600 mPas, such as between 100 and 600 mPas, or between 200 and 600 mPas when so measured. In some other embodiment, such alginates will exhibit a viscosity of between 30 and 400 mPas, such as between 30 and 300 mPas, such as between 30 and 200 mPas, or between 30 and 100 mPas when so measured. Spindle #2 can be used for viscosity measurements in the preferred viscosity range, with the above-specified equipment. In some embodiments according to the present invention, a high G type sodium alginate is used. A high G type sodium alginate means that the alginate(s) employed in the practice of the present invention possess an average of at least 50 percent adjacent G units. In some embodiments the alginate will possess an average of at least 52 percent adjacent G units; and in other embodiments such alginate will possess an average of at least 55 percent or more of adjacent G units, as such higher the content of adjacent G units may result in improved product textures.

In other embodiments according to the present invention, a high M type calcium alginate is used.

Examples of commercially available calcium alginate include Protaweld® (from FMC BioPolymer) and Kelset® from ISP Corporation.

As used herein “water-insoluble micro-dispersion” refers to system in which distributed water-insoluble solid particles of calcium alginate in micro size are dispersed in an aqueous solution.

As used herein microparticles as used herein refers to a plurality of particles with a size ranging from 0.01 micrometer to 1000 μm, typically from 0.1 or 1 micrometer to 1000 micrometer, or with a median particle size in the range of 1 micrometer to 100 micrometer, or in the range of 1 micrometer to 10 micrometer. Typically, the microdispersion is characterized by its particle size distribution, such as by the median particle size or Dn50, or the Dn90, or the Dn10.

The term “plasticizer” as used herein refers to a compound enhancing film formation used according to the present invention, which include: phthalic esters, such as dimethyl-, diethyl-, dibutyl-, and diisopropyl-phthalate; citric esters, such as triethyl-, tributyl-, acetyltriethyl- and acetyltributyl-citrate; phosphoric esters, such as triethyl-, tricresyl, and triphenyl-phosphate; alkyl lactate; glycol esters; glycerol and glycerol esters, such as glycerol triacetate also known as triacetine; sucrose esters; oils and fatty acid esters; butyl stearate; dibutyl sebacate; dibutyl tartrate; diisobutyl adipate, tributyrin; propylene glycol; a polyol, such as a sugar alcohol, such as sorbitol, mannitol, erythritol, xylitol, or glycerol (Propane-1,2,3-triol); and mixtures thereof. In some embodiments the plasticizer is not ethanol.

As used herein “the median particle size” or “Dn50” of the particles of the water-insoluble micro-dispersion of the invention is the diameter where 50 number percent of the particles have a smaller equivalent diameter and 50 number percent have a larger equivalent diameter. Typically, the Dn50 for the inventive micro-dispersion is up to 15 micrometers, more typically 14, 13, 12, 11, or 10 micrometers, such as up to 9, 8, 7, 6 micrometers, and even just up to 5 micrometers. The Dn50 for the present inventive micro-dispersion is typically 1 micrometers or more, more typically 2 micrometers or more, and most typically 3 micrometers or more.

The “Dn90” of the particles of the water-insoluble micro-dispersion of the invention refers to the diameter where 90 number percent of the particles have a smaller equivalent diameter and the other 10 number percent have a larger equivalent diameter. The equivalent particle diameter is the diameter of a sphere having the same volume as the volume of a given particle. Typically, the “Dn90” is up to 35 micrometers, typically up to 30, 28, 26, 24, 22, or 20 micrometers, more typically up to 18, 16, 14, 12, 10 or 8 micrometers; and typically, up to 12 micrometers, more typically up to 10 micrometers, and most typically up to 7 micrometers. Typically, Dn90 is 1 micrometers or more, more typically 2 micrometers or more, and most typically 4 micrometers or more.

The “Dn10” of the particles of the water-insoluble micro-dispersion of the invention refers to the diameter where 10% of the powder particles have a smaller equivalent diameter and the other 90 number percent have a larger equivalent diameter. Typically, Dn10 is 50 nanometers or more, more typically 100, 200, 300, 400, 500 nanometers or more.

These particle sizes relate to the sizes of the particles of the water-insoluble micro-dispersion of the invention after redispersion and after sieving the dispersion with a 100-160 μm sieve. The particle sizes are measured by laser diffraction particle size analysis, e.g., using a Beckman Coulter laser diffraction particle size analyzer which is commercially available from Beckman Coulter, California, such as a Beckman Coulter LS13320MW.

As used herein “capsule” and “encapsulation” is used in its normal meaning referring to the capsules used in the pharmaceutical and nutraceutical industries, for the purposes of aiding in delivering medication, nutritional components, and supplements that may have a nasty or unpleasant taste or smell or being sensitive for degradation. As such enteric capsules will only disintegrate once it reaches the target of the intestine. Capsules of the present invention is also an alternative to gelatin, where it offers the pharmaceutical and nutraceutical industry an opportunity to serve the vegans or people following a strict religious diet. In some embodiments the capsule according to the present invention do not contain significant amounts of gelatin.

A “capsule film” as used herein refers to a film formed around a capsule. Accordingly, the water-insoluble micro-dispersions of the present invention may be used to coat a capsule made of other additional or alternative components, wherein the film of the water-insoluble micro-dispersions of the present invention provides for the effect of the capsule, such as the effect needed for enteric applications.

As such “enteric oral applications” refers to the use of a medicament, a nutritional components or supplements for oral administration in capsule, where such medicament, nutritional component or supplement needs to be protected from inactivation or degradation in the acidic environment of the stomach or wherein irritation by such compounds need to be prevented, and wherein such capsule is dissolved in the intestinal canals to release the medicament, nutritional component or supplement contained therein.

As used herein “enteral administration” refers to the administration of a medicament, a nutritional component or supplement via the human gastrointestinal tract. Enteral administration involves the esophagus, stomach, and/or small and large intestines (i.e., the gastrointestinal tract). Enteral administration includes oral, sublingual, and rectal administrations. In one preferred administration route the capsules according to the present invention is for oral administration, where the enteric properties of the water-insoluble micro-dispersion according to invention is used.

A described above the present invention relates to a water-insoluble micro-dispersion comprising a) dispersed particles of calcium alginate in an amount of about 1% to about 20% by total weight of the micro-dispersion, and b) a plasticizer from at least about 30% to not more than about 200% by weight of the calcium alginate, and c) water in an amount of at least about 50% to not more than about 98% by total weight of the micro-dispersion.

In some embodiments the plasticizer is present in an amount of at least about 35% by weight of the calcium alginate, such as at least about 40%, such as at least about 45%, such as at least about 50%, such as at least about 55%, such as at least about 60%, such as at least about 65%, such as at least about 70%, such as at least about 75%, such as at least about 80%.

In some embodiments the plasticizer is present in an amount of not more than about 200% by weight of the calcium alginate, such as not more than about 195%, such as not more than about 190%, such as not more than about 185%, such as not more than about 180%, such as not more than about 175%, such as not more than about 170%, such as not more than about 165%, such as not more than about 160%, such as not more than about 155%, such as not more than about 150%.

In some embodiments the water is present in an amount of at least about 55% by total weight of the micro-dispersion, such as at least about 60%, such as at least about 65%, such as at least about 70%, such as at least about 75%, such as at least about 80%, such as at least about 85%, such as at least about 90%, such as at least about 95%, such as at least about 96%, such as at least about 98%.

In some embodiments the water is present in an amount of not more than about 97% by total weight of the micro-dispersion, such as not more than about 96%, such as not more than about 95%, such as not more than about 94%, such as not more than about 93%, such as not more than about 92%, such as not more than about 91%, such as not more than about 90%, such as not more than about 88%, such as not more than about 86%, such as not more than about 84%, such as not more than about 82%, such as not more than about 80%, such as not more than about 78%, such as not more than about 76%, such as not more than about 74%, such as not more than about 72%, such as not more than about 70%, such as not more than about 68%, such as not more than about 66%, such as not more than about 64%.

In some embodiments the dispersed particles of calcium alginate is present in an amount of at least about 2% by total weight of the micro-dispersion, such as at least about 3%, such as at least about 4%, such as at least about 5%, such as at least about 6%, such as at least about 7%, such as at least about 8%, such as at least about 9%, such as at least about 10%, such as at least about 12%, such as at least about 14%, such as at least about 16%, such as at least about 18%.

In some embodiments the dispersed particles of calcium alginate is present in an amount of not more than about 19% by total weight of the micro-dispersion, such as not more than about 18%, such as not more than about 17%, such as not more than about 16%, such as not more than about 15%, such as not more than about 14%, such as not more than about 13%, such as not more than about 12%, such as not more than about 10%, such as not more than about 9%, such as not more than about 8%, such as not more than about 7%, such as not more than about 6%, such as not more than about 5%, such as not more than about 4%, such as not more than about 3%, such as not more than about 2%.

In some embodiments the median particle size, Dn50, of the powder particles is up to 15 μm, such as up to 14, 13, 12, 11, or 10 μm, such median particle size, Dn50, being the size at which 50% of the powder particles have a smaller equivalent diameter and 50% have a larger equivalent diameter.

In some embodiments the median particle size, Dn50, of the powder particles is up to 9, such as up to 8, such up to 7, such as up to 6, such as up to 5 μm.

In some embodiments the Dn90 of the powder particles is up to 35 μm, such as up to 34, 32, 30, 28, 26, 24, 22 or 20 μm, Dn90 being the diameter where 90% of the powder particles have a smaller equivalent diameter and the other 10% have a larger equivalent diameter.

In some embodiments the Dn10 of the powder particles is 50 nm or more, Dn10 being the diameter where 10% of the powder particles have a smaller equivalent diameter and the other 90 number percent have a larger equivalent diameter.

In some embodiments the plasticizer is a polyol, such as a sugar alcohol, such as sorbitol, mannitol, erythritol, xylitol, or glycerol (Propane-1,2,3-triol), or acetate esters of glycerol selected from the class consisting of the mono-, di-, and tri-acetates of glycerol, TEC (triethyl citrate), dibutyl sebacate, and dibutyl phthalate.

In some embodiments the calcium alginate is similar or identical in properties to a calcium alginate selected from Protaweld® or Kelset®, such as FMC Textureze^{MT} 660 and FMC Protaweld™ TX 120 Alginate.

### Numbered Embodiments of the Invention

1. A water-insoluble micro-dispersion comprising
   a) dispersed particles of calcium alginate in an amount of about 1% to about 20% by total weight of the micro-dispersion, and
   b) a plasticizer from at least about 30% to not more than about 200% by weight of the calcium alginate, and
   c) water in an amount of at least about 50% to not more than about 98% by total weight of the micro-dispersion.
2. A water-insoluble micro-dispersion comprising or consisting of
   a) dispersed particles of calcium alginate in an amount of about 10% to about 30% by total weight of the micro-dispersion, and
   b) water in an amount of at least about 70% to not more than about 90% by total weight of the micro-dispersion.
3. The water-insoluble micro-dispersion according to embodiment 2, which micro-dispersion do not contain a plasticizer.
4. The water-insoluble micro-dispersion according to embodiment 1, wherein the plasticizer is present in an amount of at least about 35% by weight of the calcium alginate, such as at least about 40%, such as at least about 45%, such as at least about 50%, such as at least about 55%, such as at least about 60%, such as at least about 65%, such as at least about 70%, such as at least about 75%, such as at least about 80%.
5. The water-insoluble micro-dispersion according to any one of embodiments 1 or 4, wherein the plasticizer is present in an amount of not more than about 200% by weight of the calcium alginate, such as not more than about 195%, such as not more than about 190%, such as not more than about 185%, such as not more than about 180%, such as not more than about 175%, such as not more than about 170%, such as not more than about 165%, such as not more than about 160%, such as not more than about 155%, such as not more than about 150%.
6. The water-insoluble micro-dispersion according to any one of embodiments 1-5, wherein water is present in an amount of at least about 55% by total weight of the micro-dispersion, such as at least about 60%, such as at least about 65%, such as at least about 70%, such as at least about 75%, such as at least about 80%, such as at least about 85%, such as at least about 90%, such as at least about 95%, such as at least about 96%, such as at least about 98%.
7. The water-insoluble micro-dispersion according to any one of embodiments 1-6, wherein the water is present in an amount of not more than about 97% by total weight of the micro-dispersion, such as not more than about 96%, such as not more than about 95%, such as not more than about 94%, such as not more than about 93%, such as not more than about 92%, such as not more than about 91%, such as not more than about 90%, such as not more than about 88%, such as not more than about 86%, such as not more than about 84%, such as not more than about 82%, such as not more than about 80%, such as not more than about 78%, such as not more than about 76%, such as not more than about 74%, such as not more than about 72%, such as not more than about 70%, such as not more than about 68%, such as not more than about 66%, such as not more than about 64%.
8. The water-insoluble micro-dispersion according to any one of embodiments 1-7, wherein the dispersed particles of calcium alginate is present in an amount of at least about 2% by total weight of the micro-dispersion, such as at least about 3%, such as at least about 4%, such as at least about 5%, such as at least about 6%, such as at least about 7%, such as at least about 8%, such as at least about 9%, such as at least about 10%, such as at least about 12%, such as at least about 14%, such as at least about 16%, such as at least about 18%.
9. The water-insoluble micro-dispersion according to any one of embodiments 1-8, wherein the dispersed particles of calcium alginate is present in an amount of not more than about 19% by total weight of the micro-dispersion, such as not more than about 18%, such as not more than about 17%, such as not more than about 16%, such as not more than about 15%, such as not more than about 14%, such as not more than about 13%, such as not more than about 12%, such as not more than about 10%, such as not more than about 9%, such as not more than about 8%, such as not more than about 7%, such as not more than about 6%, such as not more than about 5%, such as not more than about 4%, such as not more than about 3%, such as not more than about 2%.
10. The water-insoluble micro-dispersion according to any one of embodiments 1-9, wherein the median particle size, Dn50, of the powder particles is up to 10 μm, such median particle size, Dn50, being the size at which 50% of the powder particles have a smaller equivalent diameter and 50% have a larger equivalent diameter.
11. The water-insoluble micro-dispersion of embodiment 10, wherein the median particle size, Dn50, of the powder particles is up to 9, such as up to 8, such up to 7, such as up to 6, such as up to 5 μm.
12. The water-insoluble micro-dispersion of any one of embodiments 1-11, wherein Dn90 of the powder particles is up to 20 μm, Dn90 being the diameter where 90% of the powder particles have a smaller equivalent diameter and the other 10% have a larger equivalent diameter.
13. The water-insoluble micro-dispersion of any one of embodiments 1-12, wherein Dn10 of the powder particles is 50 nm or more, Dn10 being the diameter where 10% of the powder particles have a smaller equivalent diameter and the other 90 number percent have a larger equivalent diameter.
14. The water-insoluble micro-dispersion of any one of embodiments 1, 4-13, wherein the plasticizer is a polyol, such as a sugar alcohol, such as sorbitol, mannitol, erythritol, xylitol, or glycerol (Propane-1,2,3-triol), or acetate esters of glycerol selected from the class consisting of the mono-, di-, and tri-acetates of glycerol, TEC (triethyl citrate), dibutyl sebacate, and dibutyl phthalate.
15. The water-insoluble micro-dispersion of any one of embodiments 1-14, wherein the calcium alginate is similar or identical in properties to a calcium alginate selected from Protaweld® or Kelset®, such as FMC Textureze^{MT} 660 and FMC Protaweld™ TX 120 Alginate.
16. A method for the preparation of a water-insoluble micro-dispersion comprising the steps of combining dispersed particles of calcium alginate in an amount of about 1% to about 20% by total weight of the micro-dispersion with a plasticizer from at least about 30% to not more than about 200% by weight of the calcium alginate, and water in an amount of at least about 50% to not more than about 98% by total weight of the micro-dispersion.
17. A method for the preparation of a water-insoluble micro-dispersion comprising the steps of combining dispersed particles of calcium alginate in an amount of about 10% to about 30% by total weight of the micro-dispersion with water in an amount of at least about 70% to not more than about 90% by total weight of the micro-dispersion.
18. A process for coating a dosage form comprising the steps of
   a) preparing the water-insoluble micro-dispersion as defined in any one of embodiments 1-15,
   b) optionally adding a further film forming aid or adjuvants to form an aqueous coating composition and
   c) coating a dosage form with the aqueous coating composition.
19. Use of a water-insoluble micro-dispersion according to any one of embodiments 1-15 for encapsulation of a drug, a nutritional or food supplement, or a combination thereof.
20. Use according to embodiment 19 for encapsulation of a drug, a nutritional or food supplement, or a combination thereof intended for enteral administration, such as enteric oral applications.
21. A capsule film made from the water-insoluble micro-dispersion of any one of embodiments 1-15.
22. The capsule film according to embodiment 21, which film has a thickness of from about 20 μm to about 200 μm, such as from about 40 μm to about 150 μm, such as from about 50 μm to about 120 μm.
23. The capsule film according to any one of embodiments 21 and 22, which film is at least 90% dissolved within 10 min to about 40 min, such as within about 20 min to about 30 min in a United States Pharmacopoeia (USP) phosphate buffer pH 6.8.
24. A capsule comprising a capsule film as defined in any one of embodiments 21-23, further comprising a drug or a nutritional or food supplement or a combination thereof.
25. A process for producing capsule film comprising the steps of providing the water-insoluble micro-dispersion of any one of embodiments 1 to 15,
   pre-heating moulding pins to a temperature higher than the aqueous composition, dipping the pre-heated moulding pins into the water-insoluble micro-dispersion, forming a film on said moulding pins by withdrawing said pins from said water-insoluble micro-dispersion, and drying the film on the moulding pins.

### EXPERIMENTAL SECTION

### Examples 1

80 g of calcium alginate was milled in a ball mill PM400 with 400 g of 2 mm balls at 400 rpm for 60 min (with 20 min pause after 20 min of milling). The balls were removed from the dispersion by sieving with a 1000 μm sieve.

Then 16.6 g of calcium alginate was milled together with 283.4 g of water in a ball mill PM400 with 410 g of 0.5 mm balls at 400 rpm for 60 min (with 20 min pause after 20 min of milling). Then additional 800 g of 0.5 mm balls was added and milled for another 20 min.

The balls were removed from the dispersion by sieving the dispersion with a 100-160 μm sieve. The particle size distribution (based on volume) was measured with a Beckman Coulter LS13320MW using the universal liquid module:

| d50 | 4 μm |
|---|---|
| <2 μm | 33% |
| <5 μm | 55% |
| <10 μm | 75% |
| <20 μm | 89% |
| <50 μm | 99.2% |

The micro-dispersion was split and glycerol (plasticizer, 50 and 100 wt.-% based on polymer) was added.

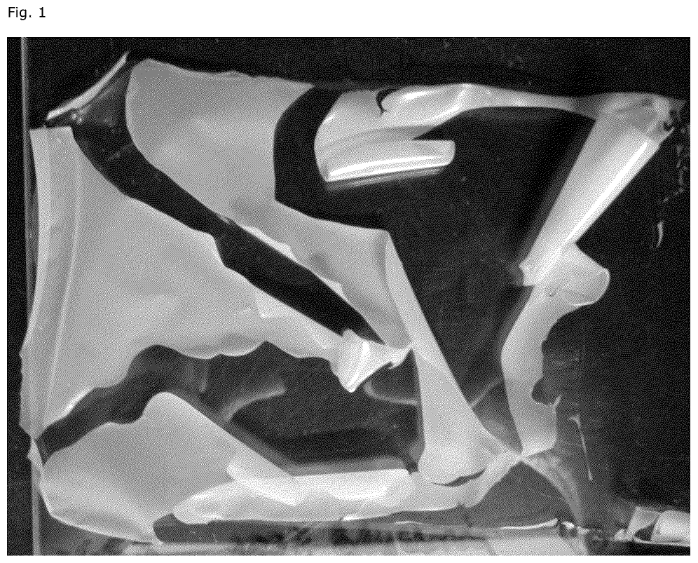

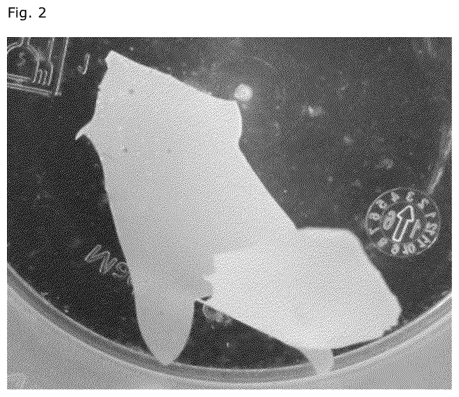

2 films were casted on a glass plate and dried for 2 h at 40° C. Two thin but firm and flexible films were obtained (FIGS. 1 and 2). In one experiment films were added to water and stored for 2 weeks at RT (FIG. 2). No sign of film dissolving was seen.

### Examples 2

A dispersion of particles containing calcium alginate was prepared as under example 1.

Glycerol (plasticizer, 50 wt. % based on polymer) was added to the dispersion.

A film was casted on a glass plate and dried for 2 h at 40° C. A thin but firm and flexible films was obtained.

A piece of the film (1 cm×1 cm of 68 μm thickness) was shaken in 15 g of 0.1N HCl for 2 h in an incubator at 37° C. at 10 rpm. The film didn't dissolve (Buffer A, table 1). Then the film was rinsed with DI water and transferred to 30 g of United States Pharmacopoeia (USP) phosphate buffer PH 6.8. The film dissolved within 30 min (Buffer B, table 1).

| Time (min) | Buffer | Result |
|---|---|---|
| 5 | A | not dissolved |
| 10 | A | not dissolved |
| 15 | A | not dissolved |
| 20 | A | not dissolved |
| 30 | A | not dissolved |
| 45 | A | not dissolved |
| 60 | A | not dissolved |
| 75 | A | not dissolved |
| 90 | A | not dissolved |
| 105 | A | not dissolved |
| 120 | A | not dissolved |
| +5 | B | not dissolved |
| +10 | B | not dissolved |
| +15 | B | not dissolved |
| +20 | B | not dissolved |
| +30 | B | completely dissolved |
| +45 | B | completely dissolved |
| +60 | B | completely dissolved |
| +75 | B | completely dissolved |
| +90 | B | completely dissolved |
| +105 | B | completely dissolved |
| +120 | B | completely dissolved |

### Examples 3

Milling was conducted with a Retsch PM400 mill using 2 grinding beakers.

Each milling beaker was filled with 22.3 g of Calcium alginate (H340100GV, dry content about 87%), 77.7 g of water and 900 g of grinding balls (2 mm size).

Milling was conducted at 400 rpm for 10 min, followed by a pause of 45 min followed by a second milling of 10 min using opposite milling direction.

The content of both grinding beakers was united and 200 g of water was added. Then the dispersion was sieved through a 1000 μm sieve to recover the grinding balls.

The particle size distribution of the dispersion (based on volume) is as follows with a Beckman Coulter LS13320MW using the universal liquid module:

| d50 | 11 μm |
|---|---|
| d90 | 33 μm |
| <2 μm | 5% |
| <5 μm | 20% |
| <10 μm | 45% |
| <20 μm | 74% |
| <30 μm | 87% |
| <40 μm | 94% |

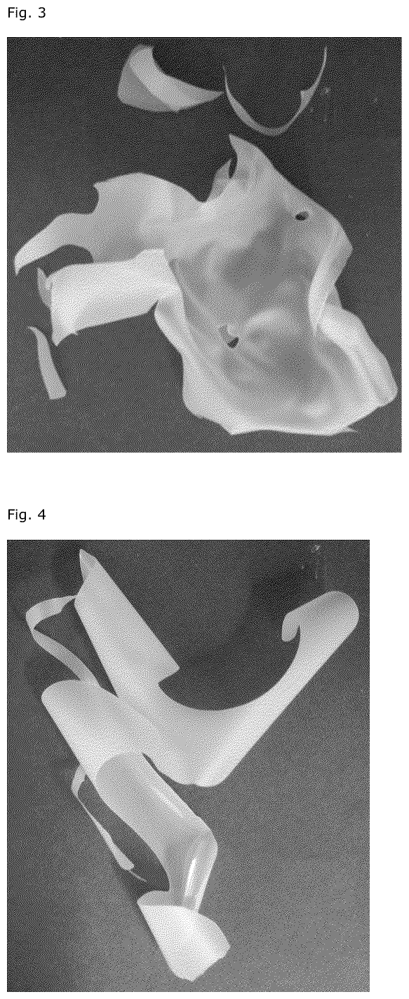

Then the dispersion was casted on a glass plate and dried for 24 h at 22° C. forming a rigid film (FIGS. 3 and 4). Dry film separated from glass plate is seen in FIG. 3.

Then the 11. 15% dispersion was further diluted to 9.15 wt-% with water and casted on a glass plate and dried for 24 h at 22° C. forming a slightly flexible film.

Dry 9.15% film separated from glass plate is seen in FIG. 4.

1. A water-insoluble micro-dispersion comprising
a) dispersed particles of calcium alginate in an amount of about 1% to about 20% by total weight of the micro-dispersion, and
b) a plasticizer from at least about 30% to not more than about 200% by weight of the calcium alginate, and
c) water in an amount of at least about 50% to not more than about 98% by total weight of the micro-dispersion.

2. A water-insoluble micro-dispersion comprising:
a) dispersed particles of calcium alginate in an amount of about 10% to about 30% by total weight of the micro-dispersion, and
b) water in an amount of at least about 70% to not more than about 90% by total weight of the micro-dispersion.

3. The water-insoluble micro-dispersion according to claim 2, wherein the micro-dispersion does not contain a plasticizer.

4. The water-insoluble micro-dispersion according to claim 1, wherein the plasticizer is present in an amount of at least about 80% by weight of the calcium alginate.

5. The water-insoluble micro-dispersion according to claim 1, wherein the plasticizer is present in an amount of not more than about 150% by weight of the calcium alginate.

6. The water-insoluble micro-dispersion according to claim 1, wherein water is present in an amount of at least about 98% by total weight of the micro-dispersion.

7. The water-insoluble micro-dispersion according to claim 1, wherein the water is present in an amount of not more than about 64% by total weight of the micro-dispersion.

8. The water-insoluble micro-dispersion according to claim 1, wherein the dispersed particles of calcium alginate are present in an amount of at least about 18% by total weight of the micro-dispersion.

9. The water-insoluble micro-dispersion according to claim 1, wherein the dispersed particles of calcium alginate are present in an amount of not more than about 2% by total weight of the micro-dispersion.

10. The water-insoluble micro-dispersion according to claim 1, wherein the median particle size, Dn50, of the powder particles is up to 10 μm, such median particle size, Dn50, being the size at which 50% of the powder particles have a smaller equivalent diameter and 50% have a larger equivalent diameter.

11. (canceled)

12. The water-insoluble micro-dispersion of claim 1, wherein Dn90 of the powder particles is up to 20 μm, Dn90 being the diameter where 90% of the powder particles have a smaller equivalent diameter and the other 10% have a larger equivalent diameter.

13. The water-insoluble micro-dispersion of claim 1, wherein Dn10 of the powder particles is 50 nm or more, Dn10 being the diameter where 10% of the powder particles have a smaller equivalent diameter and the other 90 number percent have a larger equivalent diameter.

14. The water-insoluble micro-dispersion of claim 1, wherein the plasticizer is a polyol or acetate esters of glycerol selected from mono-, di-, and tri-acetates of glycerol, TEC (triethyl citrate), dibutyl sebacate, and dibutyl phthalate.

15. (canceled)

16. A method for the preparation of a water-insoluble micro-dispersion comprising combining dispersed particles of calcium alginate in an amount of from about 1% to about 20% by total weight of the micro-dispersion with a plasticizer of from at least about 30% to not more than about 200% by weight of the calcium alginate, and water in an amount of at least about 50% to not more than about 98% by total weight of the micro-dispersion.

17. (canceled)

18. A process for coating a dosage form comprising, wherein the process comprises:
a) preparing the water-insoluble micro-dispersion as defined in claim 1, and
b) coating a dosage form with the aqueous coating composition.

19. A process for preparing an encapsulated drug, nutritional supplement or food supplement, wherein the process comprises encapsulating a drug, nutritional supplement or food supplement in a water-insoluble micro-dispersion according to claim 1.

20. (canceled)

21. A capsule film, wherein the capsule film comprises a dried water-insoluble micro-dispersion of claim 1.

22. (canceled)

23. The capsule film according to claim 21, wherein film is at least 90% dissolved within 10 min to about 40 min, such as within about 20 min to about 30 min in a United States Pharmacopoeia (USP) phosphate buffer pH 6.8.

24. A capsule, wherein the capsule comprises:
a capsule film as defined in claim 21, and
a drug or a nutritional or food supplement or a combination thereof.

25. A process for producing capsule film comprising:
providing the water-insoluble micro-dispersion of claim 1,
pre-heating moulding pins to a temperature higher than the aqueous composition,
dipping the pre-heated moulding pins into the water-insoluble micro-dispersion,
forming a film on said moulding pins by withdrawing said pins from said water-insoluble micro-dispersion, and
drying the film on the moulding pins.
